(19) United States
(12) Patent Application Publication
(10) Pub. No.: US 2022/0134012 A1
(43) Pub. Date: May 5, 2022

(54) DRUG DELIVERY DEVICE WITH MEANS FOR DETERMINING EXPELLED DOSE

(71) Applicant: Novo Nordisk A/S, Bagsvaerd (DK)

(72) Inventors: Henrik Bengtsson, Taastrup (DK); Nikolaj Eusebius Jacobsen, Soeborg (DK); Ebbe Kiilerich, Copenhagen NV (DK); Per Einar Pontus Holm, Malmoe (SE)

(21) Appl. No.: 17/433,479

(22) PCT Filed: Feb. 27, 2020
(86) PCT No.: PCT/EP2020/055099

§ 371 Date: Aug. 24, 2021

(30) Foreign Application Priority Data
Feb. 27, 2019 (EP) 19159673.3

(51) Int. Cl. A61M 5/315

(52) U.S. Cl. CPC A61M 5/31568; A61M 5/31526; A61M 2005/2411; A61M 5/31585; A61M 5/31511

(57) ABSTRACT

The present invention provides a drug delivery device (1) of the type in which a piston rod (10, 110) extends along a reference axis and is forced to rotate in an expelling direction about the reference axis during a dose expelling event to cause an expelling of drug from a drug reservoir (30, 130), and a resilient ratchet element (12) is operatively coupled with the piston rod (10, 110) and undergoes a deflecting motion comprising elastic deformation and recovery in response to an expelling of a predetermined dose increment, which deflecting motion occasions a velocity fluctuation of the piston rod (10, 110), comprising: a rotator module (50, 150) operatively coupled with the piston rod (10, 110) and configured to rotate about the reference axis against a rotation resisting force in response to a rotation of the piston rod (10, 110) in the expelling direction, the rotator module (50, 150) comprising a sensor system (70, 71; 170, 183; 270, 183) for monitoring a torque transferred by the piston rod (10, 110) during the dose expelling event, wherein the sensor system (70, 71; 170, 183; 270, 183) is configured to, from the monitored torque, identify occurrences of velocity fluctuation of the piston rod (10, 110) attributable to the deflecting motion of the resilient ratchet element 15 (12).

### FIELD OF THE INVENTION

The present invention relates generally to drug delivery devices, and more specifically to medical injection devices having means for determining the size of an expelled dose.

### BACKGROUND OF THE INVENTION

In the diabetes care segment parenteral drug administration carried out using a traditional vial and syringe system is increasingly being substituted by administration using a pen injection device. Pen injection devices are particularly convenient in that they allow the user to perform a dosed injection from a prefilled drug reservoir without first having to manually transfer the particular dose from one reservoir (the vial) to another (the syringe).

Predominantly, two types of pen injection devices are available, durable injection devices being capable of delivering one or more doses of drug from a prefilled drug cartridge which can be loaded into the device before use and replaced after exhaustion, and disposable injection devices being capable of delivering one or more doses of drug from a prefilled and non-exchangeable drug cartridge. Each of these types of pen injection devices are, or may in principle be, realised in various sub-types, such as e.g. single shot devices adapted to deliver only one dose from a drug cartridge, multi-shot devices capable of delivering a plurality of doses from a drug cartridge, manual devices, where the user provides the force needed for injection, automatic devices having a built-in energy source releasable to occasion the injection, fixed dose devices adapted to deliver the same predetermined dose of drug at each injection event, variable dose devices offering delivery of different doses of drug, settable by the user, etc.

As the labels suggest a durable injection device is intended for use over a considerable period of time during which multiple drug cartridges are exhausted and replaced, whereas a disposable injection device is intended for use until its dedicated drug cartridge is exhausted, after which the entire injection device is discarded.

In the treatment of diabetes it is advisable to keep a log of the administered doses of a particular drug (e.g. insulin or glp-1), including the respective times of dose administration. Some injection devices accordingly offer electronic dose capturing and the opportunity to review dose related information on a digital display.

As an example, U.S. Pat. No. 6,277,099 B1 (Becton, Dickinson and Company) discloses an electronic medication delivery pen, wherein a dialled dose is detected by a piezoelectric sensor arrangement, activated in response to rotation of a user manipulable dose knob, and displayed on a liquid crystal display. The medication delivery pen also comprises a memory function, which together with the liquid crystal display provides an operable interface for conveying the dose size and the time of the last five injections.

However, such type of construction is relatively expensive and not economically viable as a disposable injection device solution.

Marketed disposable injection pens such as FlexPen® and FlexTouch® by Novo Nordisk A/S offer verification of an on-going dose delivery in the form of audible clicks produced by resilient ratchet arms. The clicks are produced as a result of a repeated energy exchange between the piston rod system and the resilient ratchet arms as the piston rod moves and the resilient ratchet arms are forced to undergo elastic deformation and recovery in response thereto. In these injection pens each such click reflects a single unit of medicament expelled from the reservoir.

WO 2007/107564 (Novo Nordisk A/S) discloses an external add-on module for attachment to a pen injection device, which add-on module comprises a miniature microphone capable of picking up mechanical click sounds. The add-on module is adapted to be attached to an exterior housing surface of the pen injection device for detection of clicks produced by the dose expelling mechanism, and the size of an expelled dose can then be estimated by counting the number of clicks detected.

While the above solution enables an automatic registration of the expelled dose it does require an additional component in the system which the user must handle and which when attached to the pen injection device leads to a noticeable asymmetry in terms of both physical appearance and weight distribution that some users consider undesirable.

Co-pending International application No. PCT/EP2018/074853 discloses, i.a., an injection device capable of estimating an expelled dose of drug by use of an integrated force sensor. The solution exploits the fact that the repeated energy exchange between the piston rod system and the click-producing resilient ratchet arms during dose expelling is reflected in accompanying velocity fluctuations of the advancing piston rod, and the sensor is thus arranged to measure resultant variations in the axial force applied by the piston rod to a piston washer during drug expelling, as the number of such variations accordingly is indicative of the expelled dose.

While the integrated force sensor cancels the need for an additional system component and enables an at least substantially symmetric injection device the axial force variations occurring as an effect of the deflecting ratchet arms are relatively small and entail certain requirements to the size of the axial force applied by the piston rod as well as the axial rigidity of the dose expelling system in order to achieve a satisfactory signal-to-noise ratio.

### SUMMARY OF THE INVENTION

It is an object of the invention to eliminate or reduce at least one drawback of the prior art, or to provide a useful alternative to prior art solutions.

In particular, it is an object of the invention to provide a solution for accurately and reliably detecting clicks produced by a resilient ratchet arm in a drug delivery device to thereby enable automatic estimation of an expelled dose of drug.

It is a further object of the invention to provide such a solution which may be cost-effectively implemented in a disposable drug delivery device, e.g. a disposable injection device.

It is a further object of the invention to provide such a solution which requires a minimum of user handling and which renders a symmetric configuration of the injection device possible.

It is a further object of the invention to provide an injection device having means for automatic estimation of an expelled dose of drug which is accurate and reliable, operating with a high signal-to-noise ratio.

In the disclosure of the present invention, aspects and embodiments will be described which will address one or more of the above objects and/or which will address objects apparent from the following text.

The present invention is relevant to the type of drug delivery device, such as e.g. the above mentioned FlexPen® and FlexTouch® injection devices, which comprises a piston rod extending along a reference axis and being forced to rotate in an expelling direction about said reference axis during a dose expelling event to cause a dose of a medical substance to be expelled from a substance reservoir, and a resilient ratchet element being operatively coupled with the piston rod, e.g. via a piston rod rotator, or drive element, and configured to undergo a deflecting motion in response to an expelling of a predetermined dose increment, i.e. a predetermined volume of the medical substance, where the deflecting motion comprises a first part motion which decelerates the piston rod and a second part motion which accelerates the piston rod and produces a click.

In such type of drug delivery device the deflecting motion of the resilient ratchet element thus generates a feedback signal corresponding with the expelling of said predetermined dose increment by a dose expelling mechanism. The predetermined dose increment is a well-defined volume the size of which depends on the construction of the dose expelling mechanism. Hence, the predetermined volume of the medical substance that is expelled at each deflecting motion of the resilient ratchet element is preselected, or predefined, by the manufacturer.

The first part motion is a departing motion away from a base position of the resilient ratchet element and thus causes an accumulation of elastic energy therein, whereas the second part motion is a returning motion towards the base position and thus causes a release of the elastic energy accumulated during the first part motion. Due to conservation of energy the first part motion of the resilient ratchet element consequently causes a deceleration of the piston rod, as the piston rod loses kinetic energy, while the second part motion causes an acceleration of the piston rod, as it gains kinetic energy. Hence, during expelling of the predetermined dose increment the piston rod exhibits one cycle of deceleration and acceleration, i.e. a velocity fluctuation, attributable to the deflecting motion of the resilient ratchet element.

In one aspect the invention provides a drug delivery device as defined in claim 1.

A drug delivery device, such as e.g. a pen injection device, may thereby be provided, wherein the torque from the rotating piston rod during dose expelling is used to drive the rotator module and is monitored by means of a sensor system thereof, such that the velocity fluctuations of the piston rod which result from the deflecting motions of the resilient ratchet element and which are reflected by accompanying variations of the transmitted torque, can be identified for estimation of the size of the expelled dose. The sensor system may comprise a sensor producing a characteristic signal in response to a variation of the torque and may be configured to identify occurrences of velocity fluctuation of the piston rod attributable to the deflecting motion of the resilient ratchet element from signals produced by the sensor. The sensor may e.g. be configured to produce the characteristic signal in response to an increase of the torque indicative of an acceleration of the piston rod, or in response to a decrease of the torque indicative of a deceleration of the piston rod.

A monitoring of the torque is much more reliable than a monitoring of the axial force due to the rotational stiffness of the piston rod, ensuring that the signal generated by the deflection of the resilient ratchet element is transmitted through the piston rod directly to the sensor system with a high signal-to-noise ratio, regardless of the size of the torque.

The dose expelled in the course of a dose expelling event may thereby be estimated automatically from the identified occurrences of velocity fluctuation of the piston rod and the predetermined dose increment. For example, the identified occurrences of velocity fluctuation of the piston rod may be summated, and the result may be multiplied by the predetermined dose increment to obtain the expelled dose. Alternatively, a value equalling the predetermined dose increment may be assigned to each occurrence of velocity fluctuation and the assigned values may then be summated to obtain the expelled dose.

The estimation of the expelled dose may be obtained by the sensor system, e.g. comprising a sensor processor for performing calculations based on the identified occurrences of velocity fluctuation of the piston rod and a value of the predetermined dose increment pre-inputted in the sensor processor.

Hence, a drug delivery device may be provided which comprises a housing and a dose expelling mechanism, the latter comprising a piston rod adapted to rotate in an expelling direction about a reference axis during a dose expelling event to cause an expelling of drug from a drug reservoir. This may be accomplished by a threaded piston rod being advanced helically through a nut member rotationally fixed with respect to, e.g. formed integrally in, the housing. The dose expelling mechanism further comprises a resilient ratchet element operatively coupled with the piston rod and configured to undergo a deflecting motion in response to a predetermined angular displacement of the piston rod, corresponding to an expelling of a predetermined volume of the drug. The deflecting motion comprises a first part motion during which some of the rotational energy of the piston rod is converted and stored as elastic energy in the resilient ratchet element and a second part motion during which the stored elastic energy is released and converted to rotational energy of the piston rod, resulting in a jerking motion thereof. The drug delivery device further comprises a rotator module operatively coupled with the piston rod and adapted to rotate about the reference axis against a rotation resisting force in response to a rotation of the piston rod in the expelling direction, and a sensor system for monitoring a torque transmitted from the piston rod to the rotator module during the dose expelling event, where the sensor system is configured to, from the monitored torque, a) identify occurrences of velocity fluctuation of the piston rod attributable to the deflecting motion of the resilient ratchet element, b) summate the identified occurrences of velocity fluctuation of the piston rod, thereby determining a number of deflecting motions undergone by the resilient ratchet element during the dose expelling event, and c) multiply the number of deflecting motions by the predetermined dose increment to thereby estimate an expelled dose of drug. The estimation of the expelled dose may alternatively be performed e.g. by assigning to each occurrence of velocity fluctuation a value equalling the predetermined dose increment and then summating the assigned values.

Alternatively, the estimation of the expelled dose may be obtained by an external device comprising an external device processor configured to perform calculations based on relayed information regarding occurrences of velocity fluctuation of the piston rod identified by the sensor system and a value of the predetermined dose increment pre-inputted in the external device processor.

In any case, the drug delivery device may further comprise a communication interface for communicating information relating to the occurrences of velocity fluctuation of the piston rod identified by the sensor system. If the estimation of the expelled dose is obtained by the sensor system the communication interface may for example comprise means for at least one of visual, audible and tactile representation of the estimated expelled dose, e.g. a display, a loudspeaker and/or a vibrator. Alternatively, or additionally, the communication interface may comprise a transmission interface for wired or wireless transmission of data to an external device.

If the estimation of the expelled dose is obtained by the external device the communication interface may for example comprise a transmission interface for wired or wireless transmission of e.g. a value representing an accumulated number of velocity fluctuations of the piston rod identified by the sensor system during the dose expelling event. This value is then converted to a size of the expelled dose by the external device processor using the pre-inputted value of the predetermined dose increment.

The external device may be any device capable of receiving the transmitted information, and potentially performing the estimation of the expelled dose of drug based on the received information, and either displaying or forwarding the estimated expelled dose, such as e.g. a mobile phone, a tablet computer, a pc, a second drug delivery device, a diagnostic device, etc.

By estimating the expelled dose in the drug delivery device itself and indicating the result directly to the user via the communication interface it is possible to offer a self-contained device which is capable of automatically gathering and relaying the important dose related information in a relatively inexpensive manner without the need for additional system components, whereas by gathering the information in the drug delivery device and relaying the information for estimation of the expelled dose in the external device a stripped down version of the drug delivery device may be offered for combination with a more capable external device in terms of processing power, display options, etc., which external device may be configured to additionally perform a number of other, related or non-related, operations.

In some embodiments of the invention the communication interface comprises both means for at least one of visual, audible and tactile representation of the estimated expelled dose and a transmission interface for wired or wireless transmission of the estimated expelled dose to an external device, whereby the drug delivery device is capable of estimating the expelled dose from the identified occurrences of velocity fluctuation of the piston rod obtained by the sensor system and a value of the predetermined dose increment pre-inputted in the sensor processor and subsequently both relay the result directly to the user and to the external device for potential further processing.

The rotator module may in principle be arranged in any suitable position in the drug delivery device, where it can be operatively coupled with the piston rod. For example, the rotator module may be arranged between the piston rod and a wall portion of the drug reservoir which is displaced along the reference axis by the piston rod during the dose expelling event. In the case of a drug delivery device, such as an injection pen, holding a cartridge type drug reservoir having a generally cylindrical side wall surrounding a displaceable piston the rotator module may thus be arranged between the piston rod and the piston, e.g. replacing a conventional piston washer. Such arrangement has the advantages of the rotator module being easily positionable during assembly and avoiding mechanical interference with other moving parts of the dose expelling mechanism.

The rotator module is configured to rotate about the reference axis against a rotation resisting force in order to enable the sensor system to detect any torque variation stemming from a velocity fluctuation of the piston rod. The rotation resisting force may for example comprise a frictional force, and in case the rotator module is arranged between the piston rod and the wall portion of the drug reservoir which is displaced along the reference axis by the piston rod during dose expelling the frictional force may be established between a portion of the rotator module and a wall portion of the drug reservoir which is not displaced along the reference axis during dose expelling.

Hence, the rotator module may e.g. comprise a radially protruding lip structure, and the rotation resisting force may be provided by a friction interface between the lip structure and an interior surface portion of the drug reservoir. In case of the cartridge type drug reservoir the rotation resisting force may be provided by a friction interface between the lip structure and the generally cylindrical side wall. Since the rotator module is destined to rotate relative to the generally cylindrical side wall during dose expelling anyway the provision of the lip structure is a simple manner of obtaining the required rotation resisting force.

In some embodiments of the invention the rotator module is arranged between the piston rod and the wall portion of the drug reservoir which is displaced along the reference axis during dose expelling and comprises a first part being in rotational interlocking engagement with the piston rod and a second part including the lip structure and being rotationally coupled with the first part by a torque transmitting structure, and the sensor system comprises a sensor arranged on the torque transmitting structure. During a dose expelling event the rotating piston rod will thus rotate the first part and the torque transmitting structure will transmit the torque from the rotating first part to the second part which then rotates against the rotation resisting force provided by the friction interface between the lip structure and the drug reservoir. Any tangential velocity fluctuation of the rotating piston rod and the first part is thereby captured by the sensor on the torque transmitting structure as the torque transmitting structure drives the reluctant second part.

The rotator module may further comprise an intermediate part arranged between the first part and the second part, where the intermediate part carries the sensor system and the torque transmitting structure, as it may be convenient in the production and assembly setup to pre-arrange electronics on a separate component part.

In other embodiments of the invention the rotator module comprises a first part arranged concentrically with the piston rod at the distal end thereof and including an axially extending portion, and a second part carrying the lip structure and being rotationally locked to the first part, the piston rod comprises a radial protrusion configured to apply a tangential drive force to the axially extending portion, and the sensor system comprises a sensor arranged between the radial protrusion and the axially extending portion. This allows the sensor to monitor the torque transmitted from the rotating piston rod to the first part directly.

The rotator module may further comprise an intermediate part arranged substantially between the first part and the second part, where the intermediate part carries the sensor system and comprises an axially extending sensor carrier on which the sensor is arranged. Again, it may be convenient in the production and assembly setup to pre-arrange electronics on a separate component part.

The second part may comprise a cup shaped wall forming a cavity, and the intermediate part may be arranged substantially in the cavity such that the axially extending sensor carrier extends proximally therefrom. Such an arrangement is space-saving and provides a rotator module with a small axial extent, which may be attractive in case a minimisation of the axial dimension of the drug delivery device is desired.

The sensor may be a piezoelectric sensor generating an electrical signal in response to a change in the impact thereon. Such a sensor is both inexpensive in production and takes up very little space. Alternatively, the sensor may be a strain gauge, a capacitive sensor, etc.

In another aspect the invention provides a rotator module for use in a drug delivery device of the type described above.

In a further aspect the invention provides a method of detecting clicks produced by a resilient ratchet element in a drug delivery device in which a piston rod is forced to rotate about a reference axis during a dose expelling event to cause an expelling of drug and the resilient ratchet element is operatively coupled with the piston rod and undergoes a deflecting motion in response to expelling of a predetermined dose increment, which deflecting motion occasions a velocity fluctuation of the piston rod and results in a click, the method comprising: (i) monitoring a torque transferred by the piston rod during the dose expelling event, and (ii) identifying, from the monitored torque, occurrences of velocity fluctuation of the piston rod attributable to the deflecting motion of the resilient ratchet element.

Steps (i) and (ii) may for example be realised by a sensor system configured to produce a signal in response to an abrupt change of tangential velocity of the piston rod, e.g. as experienced during the second part motion of the resilient ratchet element, which causes an acceleration of the piston rod. The number of signals produced during the dose expelling event may then be used as a measure of the occurrences of velocity fluctuation of the piston rod attributable to the deflecting motion of the resilient ratchet element.

As mentioned above the number of clicks produced by the resilient ratchet element is indicative of the dose expelled during the dose expelling event. Hence, in a further aspect the invention provides a method of determining an expelled dose of drug from a drug delivery device in which a piston rod is forced to rotate about a reference axis during a dose expelling event to cause an expelling of drug and a resilient ratchet element is operatively coupled with the piston rod and undergoes a deflecting motion comprising elastic deformation and recovery in response to expelling of a predetermined dose increment, said deflecting motion occasioning a velocity fluctuation of the piston rod, the method comprising: (i) monitoring a torque transferred by the piston rod during the dose expelling event, (ii) identifying, from the monitored torque, occurrences of velocity fluctuation of the piston rod attributable to the deflecting motion of the resilient ratchet element, and (iii) calculating the expelled dose of drug from the identified occurrences of velocity fluctuation of the piston rod and the predetermined dose increment.

A resilient ratchet element as described in the present text may be or comprise a resilient click generating arm such as those present in the aforementioned FlexPen® and FlexTouch® devices. In exemplary embodiments of the invention the dose expelling mechanism includes at least two resilient ratchet arms.

For the avoidance of any doubt, in the present context the term “medical substance” is interchangeable with the term “drug” and designates a medium which is used in the treatment, prevention or diagnosis of a condition, i.e. including a medium having a therapeutic or metabolic effect in the body. Further, the terms “distal” and “proximal” denote positions at or directions along a drug delivery device, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a perspective longitudinal section view of a drug delivery device according to a first embodiment of the invention,

FIG. 2 is a perspective cross-sectional view of a proximal portion of the injection device,

FIG. 3 is an enlarged perspective longitudinal section view of a central portion of the injection device including a first exemplary embodiment of a sensor module used therein,

FIG. 4 is a perspective longitudinal section view detailing the sensor module,

FIG. 5 is a perspective cross-sectional view of the sensor module,

FIG. 6 is a graphical representation of sensor measurements during a dose expelling action,

FIG. 7 is an exploded view of an alternative sensor module,

FIG. 8 is a perspective, partially transparent, view of the arrangement of the sensor module of FIG. 7 in relation to a cartridge type drug reservoir,

FIG. 9 shows the parts of FIG. 8 in a longitudinal section view, and

FIG. 10 is a perspective, partially transparent, view of a variation of the sensor module of FIG. 7 in an arrangement similar to that of FIG. 8.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

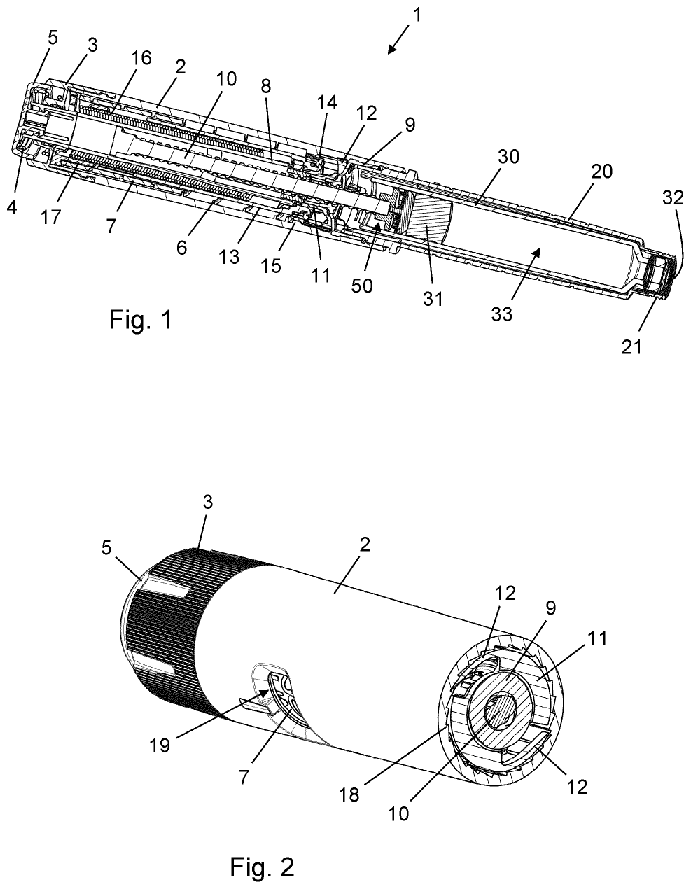

FIG. 1 is a perspective longitudinal section view of a drug delivery device according to a first embodiment of the invention, in the form of an injection pen 1. The injection pen 1 comprises a housing 2 extending along a longitudinal housing axis and a cartridge holder 20 which is at least axially fixed with respect to the housing 2 and which holds a drug cartridge 30. A nut element 9 is arranged in the housing 2 just proximally of the cartridge holder 20. The nut element 9 serves to support an axially extending piston rod 10 and to enable helical advancement of the piston rod 10 relative to the housing 2 via a threaded interface. The drug cartridge 30 has a generally cylindrical wall which extends between a proximal end and a distal end and which comprises a narrowed distal end portion. The distal end is sealed by a penetrable septum 32 and the drug cartridge 30 further comprises a piston 31 arranged in sealing contact with an interior surface of the generally cylindrical wall such that a chamber 33 is defined by the generally cylindrical wall, the piston 31 and the septum 32. The chamber 33 holds a medical substance which is accessible by insertion through the penetrable septum 32 of a hollow needle structure, e.g. a rear portion of an injection needle forming part of a pen needle unit (not shown) which further comprises a collar capable of attachment, in conventional manner, to a needle mount 21 of the cartridge holder 20. In the present embodiment the injection pen 1 is of the disposable type, and it is not possible to remove the drug cartridge 30 without damaging the injection pen 1. However, it is noted that the injection pen 1 could just as well be a durable type of device allowing for exchange of the drug cartridge 30.

The injection pen 1 is operable to set a desired dose of the medical substance to be injected and to expel the set dose through an attached injection needle. Accordingly, the injection pen 1 comprises a dose setting mechanism and a dose expelling mechanism. The dose setting mechanism comprises a user operable dose dial 3, a scale drum 7 having a plurality of dose numerals arranged thereon, a reset tube 8, a ratchet tube 13, and a torsion spring 16, and is configured to allow both dialling up and dialling down to set a dose and to adjust a set dose. The particular operation of the dose setting mechanism is similar to the operation of the dose setting system in the injection device disclosed in WO 2015/071354 and will not be described further in the present text, since the dose setting mechanism as such is irrelevant to the present invention, being concerned only with the determination of an expelled dose. For details on the operation of the dose setting mechanism reference is made to the aforementioned WO 2015/071354, particularly p. 10, I. 21-p. 15, I. 13.

In the following the various components, and the operation, of the injection pen 1 will be described based on the dose expelling functionality.

An injection button 5 is slidably arranged at the proximal end of the housing 2. The injection button 5 is axially fixed to the reset tube 8 and is biased proximally by a button spring 4. The reset tube 8 is at its distal end portion axially and rotationally coupled with the ratchet tube 13 such that a distal displacement of the reset tube 8 causes a corresponding distal displacement of the ratchet tube 13 and a rotation of the ratchet tube 13 in a dose expelling direction causes a corresponding rotation of the reset tube 8.

The torsion spring 16 extends axially along an exterior surface of the reset tube 8 and has a proximal end attached to a spring base 17 and a distal end attached to the ratchet tube 13. The spring base 17 is axially and rotationally fixed to the housing 2, and the torsion spring 16 is pre-strained during assembly of the injection pen 1, biasing the ratchet tube 13 in the dose expelling direction relative to the housing 2 (clockwise when seen from the distal end), to ensure sufficient power to expel an entire set dose regardless of its size.

The ratchet tube 13 is rotationally interlocked with the scale drum 7 via a spline interface, and the scale drum 7 is provided with an exterior helical groove which is in engagement with a helical rib 6 on an interior surface portion of the housing 2 such that a rotation of the ratchet tube 13 in the dose expelling direction causes a helical proximal displacement of the scale drum 7 in the housing 2, and a rotation of the ratchet tube 13 opposite the dose expelling direction causes a helical distal displacement of the scale drum 7 in the housing 2.

The ratchet tube 13 is at its distal end portion axially locked to a clutch 14. The clutch 14 is provided with a plurality of exterior spline elements (not visible) which in a dose setting axial position of the clutch 14 engage with corresponding housing splines 15 on an interior surface of the housing 2, thereby rotationally locking the clutch 14 to the housing 2. The clutch 14 is further provided with an interior toothed structure (not visible) configured for interaction with a flexible arm (not visible) on the ratchet tube 13 so as to ensure joint rotation of the ratchet tube 13 and the clutch 14 in the dose expelling direction.

Also, the clutch 14 is rotationally locked to a piston rod drive element 11 arranged about the piston rod 10. The piston rod 10 has an exterior threaded section and two opposite longitudinal grooves (not visible), and the piston rod drive element 11 has a central bore with two opposite protrusions (not visible), each of which engage one of the grooves to provide a rotational interlocking connection between the piston rod drive element 11 and the piston rod 10. The piston rod drive element 11 further has a pair of opposite ratchet arms 12 acting to restrict its rotational degrees of freedom relative to the housing 2, as explained below in relation to FIG. 2.

During setting of a dose the torsion spring 16 becomes further strained. In order to expel a set dose the injection button 5 is depressed against the proximal end of the housing 2. This will displace the reset tube 8 axially in the distal direction, and the reset tube 8 will slave the ratchet tube 13 and the clutch 14. As a result the clutch 14 will slide out of engagement with the housing splines 15 and begin to rotate in the dose expelling direction driven by the thereby released torsion spring 16 via its rotational connection to the ratchet tube 13.

The rotation of the ratchet tube 13 and the clutch 14 as the torsion spring 16 unwinds causes a helical proximal motion of the scale drum 7 as well as a rotation of the piston rod drive element 11 and, accordingly, of the piston rod 10. Due to the threaded interface between the piston rod 10 and the nut element 9 this will cause a helical distal advancement of the piston rod 10 into the drug cartridge 30. The distal end of the piston rod 10 is connected to a specially designed piston washer in the form of a sensor module 50, described in detail below, which as a result of the movement of the piston rod 10 forces the piston 31 into the drug cartridge 30 to thereby expel the set dose of medical substance from the chamber 33 through the attached injection needle.

FIG. 2 is a perspective view of a proximal portion of the housing 2, cross-sectioned through the nut element 9 to show that the ratchet arms 12 are axially aligned with ratchet teeth 18 arranged around an interior circumferential surface portion of the housing 2. Each ratchet arm 12 is formed as a circumferential extension of a peripheral portion of the piston rod drive element 11 and constitutes a suspended, flexible curved beam having a radially outwardly directed bias. An end portion of each ratchet arm 12 interacts with a section of the ratchet teeth 18, providing a unidirectional ratchet mechanism which prevents counter-clockwise (seen from the distal end) rotation of the piston rod drive element 11 relative to the housing 2.

In the course of the dose expelling action described above the joint rotation of the ratchet tube 13, the clutch 14, and the piston rod drive element 11 in the dose expelling direction causes each of the ratchet arms 12 to ride over a number of the ratchet teeth 18. The ratchet mechanism is configured such that two opposite ratchet teeth 18 are passed by the respective ratchet arms 12 simultaneously, and one such simultaneous passage of two opposite ratchet teeth 18 is correlated with one unit of the medical substance being expelled from the drug cartridge 30.

During the clockwise rotation of the piston rod drive element 11, as a consequence of the interaction with the ratchet teeth 18 and their respective directional bias, each of the ratchet arms 12 will undergo a deflecting motion as it passes one of the ratchet teeth 18. Observing one of the ratchet arms 12, an angular displacement of the piston rod drive element 11 corresponding to one unit of the medical substance being expelled from the drug cartridge 30 will cause the end portion of the ratchet arm 12 to firstly slide along a ratchet tooth 18 from a tooth trough base position to a tooth tip deflected position and secondly to pass the tooth tip and assume a new base position at the subsequent tooth trough. In the present context this is referred to as one deflecting motion of the ratchet arm 12, which then comprises a first part motion from the tooth trough base position to the tooth tip deflected position and a second part motion from the tooth tip deflected position to the new base position.

The movement from the tooth trough base position to the tooth tip deflected position deflects the ratchet arm 12 gradually radially inwardly, against its bias, thereby storing energy in the ratchet arm 12 and increasing the friction between the end portion of the ratchet arm 12 and the ratchet tooth 18, resulting in a momentary decrease of the speed of rotation of the piston rod drive element 11. As the end portion of the ratchet arm 12 passes the tooth tip the energy stored in the ratchet arm 12 is released, forcing the end portion of the ratchet arm 12 towards the subsequent tooth trough, and the friction is abruptly reduced, resulting in a momentary increase of the speed of rotation of the piston rod drive element 11.

This repetitive accumulation and release of energy is reflected in the torque which the piston rod 10, driven by the rotation of the piston rod drive element 11, applies to the sensor module 50, and it is consequently possible to estimate the size of an expelled dose by monitoring this torque.

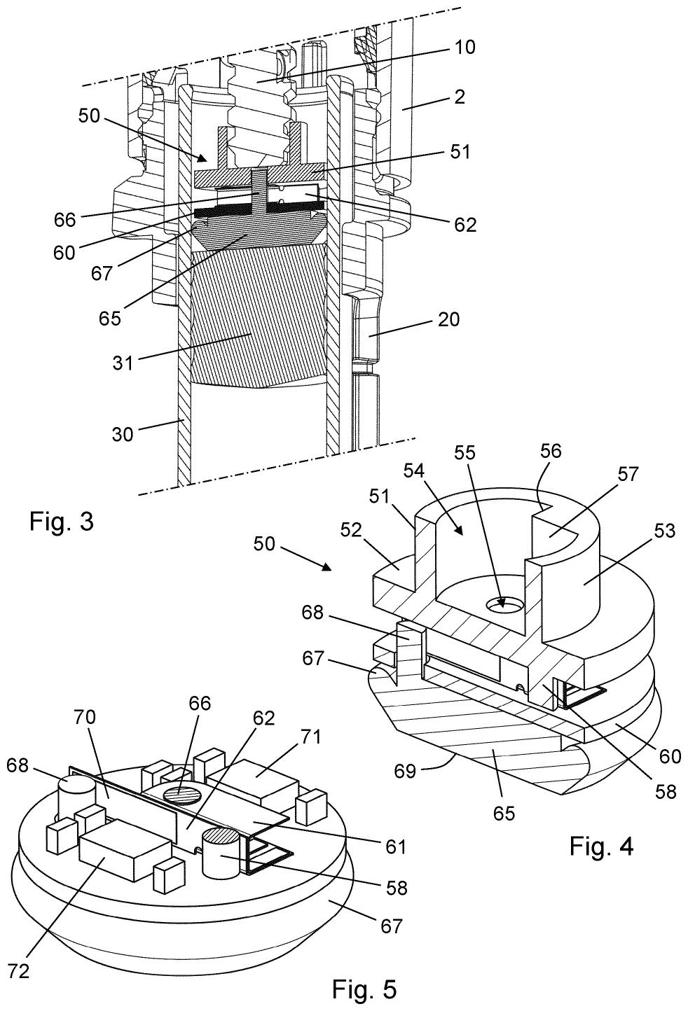

To that end the injection pen 1 comprises the sensor module 50. FIG. 3 is a close up view of a central portion of the injection pen 1, and FIG. 4 is a perspective longitudinal section view detailing the sensor module 50. As can be seen from FIG. 3 the sensor module 50 comprises a top part 51, an intermediate part 60, and a bottom part 65, stacked axially, sandwiching a sheet metal 62, and aligned radially by an alignment member 66 extending proximally from the bottom part 65. The sensor module 50 is arranged between the piston rod 10 and the piston 31 in such a way that the top part 51 axially abuts and rotationally engages a distal end portion of the piston rod 10, and the bottom part 65 abuts a proximal surface of the piston 31. The bottom part 65 is formed of a rubber material and has a circumferential friction lip 67 in contact with the generally cylindrical wall of the drug cartridge 30

The top part 51 comprises a circular base 52 with a central hole 55 in which the alignment member 66 resides in the assembled state of the sensor module 50. A tower 53 protrudes proximally from the circular base 52 and forms a cavity 54 for reception of the distal end portion of the piston rod 10. The tower 53 has an internal configuration which comprises a first axially extending planar contact surface 56 and a second axially extending planar contact surface 57 at right angles to the first axially extending planar contact surface 56. These two contact surfaces 56, 57 conform with similar contact surfaces at the distal end portion of the piston rod 10 so as to provide a rotational fixation of the sensor module 50 to the piston rod 10. The top part 51 further comprises an eccentrically positioned descending peg member 58 serving to transmit the torque from a rotating top part 51 to the intermediate part 60, as described in the below.

The bottom part 65 comprises, apart from the alignment member 66, an eccentrically positioned ascending peg member 68, the role of which will be clear from the below, and a planar bottom surface 69 for abutment with the piston 31.

The intermediate part 60 carries a plurality of components, as schematically illustrated in FIG. 5 which shows the sensor module 50 in a perspective cross-sectional view. Arranged about the alignment member 66, and held in position thereby, is a bracket 61 which supports a portion of the sheet metal 62 that extends transversally between the descending peg member 58 and the ascending peg member 68. A piezoelectric sensor 70 is printed on the sheet metal 62 and electrically connected (not shown) with a processor 71 which in turn is electrically connected (not shown) with a loudspeaker 72. Power for the electrical components is provided by a battery (not shown), e.g. in the form of a printed battery or a small button cell.

In use, during a dose expelling event, as the piston rod 10 rotates through the nut element 9 and advances axially relative to the housing 2 and the cartridge holder 20 the top part 51 undergoes a similar rotation, as the torque from the piston rod 10 is transmitted to the tower 53 via the second axially extending planar contact surface 57. The top part 51 further undergoes a similar axial displacement as the piston rod 10, being pushed in the distal direction thereby, and since the sensor module 50 as a whole is axially incompressible so does the bottom part 65 and the proximal surface of the piston 31.

The rotation of the top part 51 is transmitted to the intermediate part 60 by interaction between the descending peg member 58 and the metal sheet 62, the latter being supported by the bracket 61 along the majority of its length such that the majority of the metal sheet 62 substantially functions as a rigid beam and a minor portion functions as a suspended beam. As the descending peg member 58 applies a force to the sheet metal 62 and the intermediate part 60 resultantly rotates about the alignment member 66 the suspended portion of the metal sheet 62 abuts the ascending peg member 68 and transmits the force to thereto, thus causing the bottom part 65 to rotate along with the intermediate part 60 and the top part 51.

Due to the interaction between the friction lip 67 and the drug cartridge 30 the rotation of the bottom part 65 is resisted sufficiently to slightly bend the suspended portion of the metal sheet 62 when the top part 51 rotates. The piezoelectric sensor 70 is arranged, e.g. printed or mounted, on the suspended portion of the metal sheet 62 and is capable of registering changes to the contact force between the metal sheet 62 and the ascending peg member 68 as the sensor module 50 rotates.

More specifically, the piezoelectric sensor 70 generates a signal in response to a sudden deflection. As explained above, during dose expelling the piston rod is repeatedly decelerated and accelerated due to the interaction between the ratchet arms 12 and the ratchet teeth 18, and this is reflected in the torque which the piston rod 10 applies to the top part 51 and the top part 51 transmits to the bottom part 65. As the ratchet arms 12 respectively undergo the first part motion of the deflecting motion, urged by the ratchet teeth 18, during which they accumulate elastic energy the torque applied by the piston rod 10 gradually decreases, and when the ratchet arms 12 pass the tooth tips and undergo the second part motion driven by the sudden release of the accumulated elastic energy the torque abruptly increases.

The abrupt torque increase causes a small jerking motion of the top part 51 and consequently an abrupt force increase between the ascending peg member 68 and the sheet metal 62, resulting in an excitation of the piezoelectric sensor 70. Accordingly, as the dose expelling progresses the piezoelectric sensor 70 generates a signal each time the ratchet arms 12 undergo the second part motion, and the pulsating torque can thus be captured and used to estimate the size of the dose expelled.

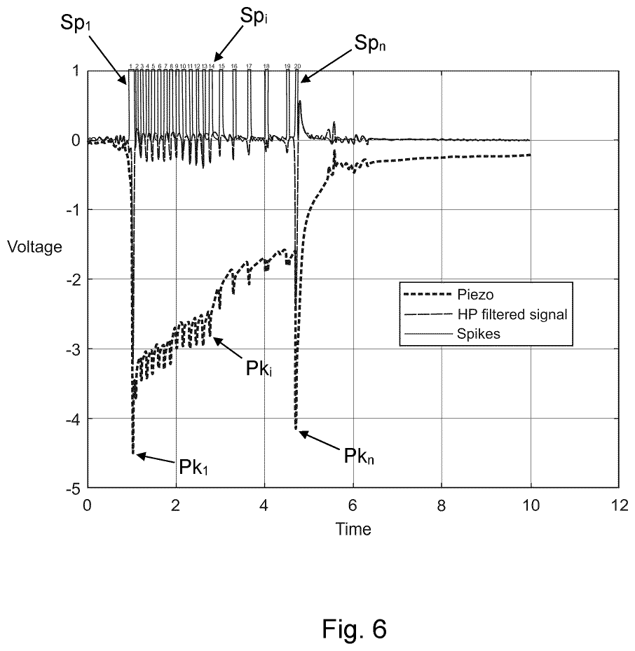

FIG. 6 illustrates the torque variation over time during an expelling of n units of the medical substance from the drug cartridge 30. As can be seen from the sensor output, represented by the narrowly dotted curve, the torque fluctuates as the dose expelling progresses, delimited by a first peak, Pk₁, and a last peak, Pkₙ. Each individual peak, Pkᵢ, reflects a deflecting motion of both ratchet arms 12, and since each deflecting motion of both ratchet arms 12 corresponds to one unit of the medical substance expelled from the drug cartridge 30 the total expelled dose can be estimated by counting the peaks, Pkᵢ, exhibited during the dose expelling event.

The sensor measurements may be filtered using any of a number of suitable methods, such as e.g. high-pass, low-pass, band-pass, matched, and adaptive matched filtering, to maximise the signal-to-noise ratio. In FIG. 6 the widely dotted curve shows the result of a high-pass filtering. By running this result through a simple spike detection algorithm the sensor signals can be transformed to easily distinguishable spikes, delimited by a first spike, Sp₁, which corresponds to the first peak, Pk₁, and a last spike, Spₙ, which corresponds to the last peak, Pkₙ. Since each individual spike, Spᵢ reflects one unit of the medical substance expelled from the drug cartridge 30 the total expelled dose can be estimated by counting the spikes, Spᵢ produced by the spike detection algorithm.

The sensor signals 70 are captured and processed by the processor 71 and the final estimated value of n is announced through the loudspeaker 72 by a speech synthesized voice, e.g. at a predetermined time following detected completion of the dose expelling event.

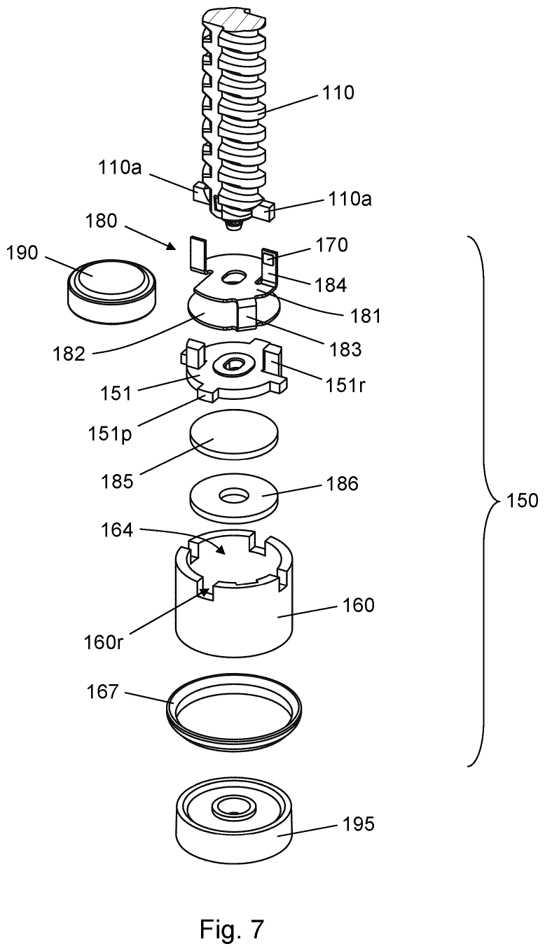

FIG. 7 is an exploded view of an alternative sensor module 150 for use in a drug delivery device according to a second embodiment of the invention. The drug delivery device is principally of the same type as the injection pen 1, i.e. a device in which a piston rod is forced to rotate about a longitudinal axis in a dose expelling direction during a dose expelling event to cause an expelling of drug from a drug reservoir and a resilient ratchet element is operatively coupled with the piston rod and undergoes a deflecting motion comprising elastic deformation and recovery in response to an expelling of a predetermined dose increment, occasioning a velocity fluctuation of the piston rod, and the principle of dose detection is basically the same as for the injection pen 1. Hence, FIG. 7 only shows the components that are functionally different from the first embodiment.

The sensor module 150 comprises a PCB 180 having a top sheet 181, a bottom sheet 182 spaced apart from the top sheet 181 along a reference axis, and a bridge 183 connecting the top sheet 181 and the bottom sheet 182 and carrying a processor (not visible). A battery 190 providing electrical power is arranged between the top sheet 181 and the bottom sheet 182. The top sheet 181 is formed to provide two diametrically opposite axially extending strips 184, one of which acting as a sensor support carrying a piezoelectric sensor 170.

The PCB 180 with the battery 190, as well as a Bluetooth module 185 and an antenna 186, are arranged in a cavity 164 of a module housing 160 having a proximal surface with a plurality of recesses 160r adapted to receive an equivalent plurality of radial protuberances 151p of a reaction member 151 in a rotationally interlocked relationship, the reaction member thus acting as a lid for the module housing 160. The reaction member 151 comprises two diametrically opposite axially extending reaction plates 151r, each reaction plate 151r supporting one of the strips 184. A friction ring 167 is fixedly attached around an exterior surface portion of the module housing 160.

FIG. 7 further shows a piston washer 195 and a distal portion of a piston rod 110 employed in the drug delivery device according to the second embodiment of the invention. The piston rod 110 is provided with two diametrically opposite radial protrusions 110a at its distal end portion configured to interact with the strips 184 and the reaction plates 151r, as described below.

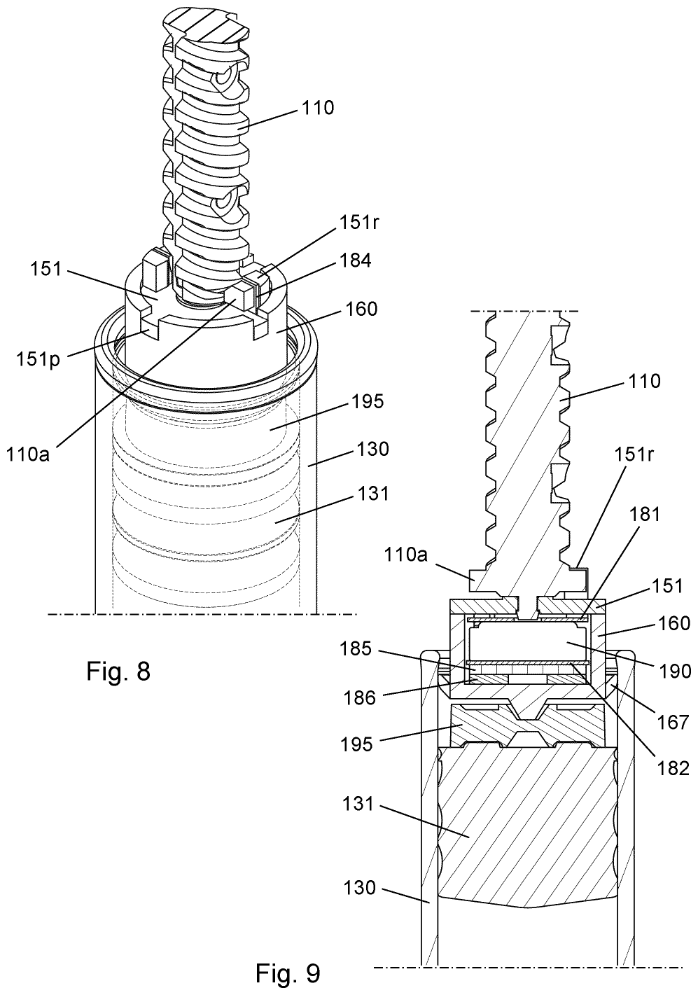

FIGS. 8 and 9 are, respectively, a perspective, partially transparent, view and a longitudinal section view of the components of FIG. 7 as used with a drug cartridge 130 in the drug delivery device according to the second embodiment of the invention. For the sake of clarity other drug delivery device components are omitted from these views. The figures show a functional state of a dose estimation system based on the sensor module 150 in which the radial protrusions 110a are aligned with the strips 184 and the reaction plates 151r. The piston washer 195 is a conventional type spacer device which is placed between the module housing 160 and a piston 131 in the drug cartridge 130 to ensure an axisymmetrical distribution of the driving force applied to the piston 131 by the piston rod 110.

During a dose expelling event, as the piston rod 110 rotates the radial protrusions 110a transmit a drive torque from the piston rod 110 to the reaction member 151 by application of a tangential force to the reaction plates 151r. Due to the rotationally interlocked connection between the reaction member 151 and the module housing 160 both will rotate along with the piston rod 110 and slave the components housed in the cavity 164. The friction ring 167 is in sealing contact with an interior surface portion of the drug cartridge 130, so the rotation of the sensor module 150 occurs against a frictional resistance, similar to that provided by the friction lip 67 in the first embodiment of the invention.

As the dose expelling progresses the piezoelectric sensor 170, which is in physical contact with one of the radial protrusions 110a, will sense the torque variations stemming from the velocity fluctuations of the piston rod 110 as jerking tangential force impacts and will generate a signal at each sudden force increase indicating passage of a tooth tip by a ratchet arm (neither shown), in accordance with the above described.

The signals are relayed to the processor on the bridge 183 and may be processed as described above in connection with the first embodiment of the invention to estimate the size of the expelled dose. The estimated dose value is then transmitted wirelessly by the Bluetooth module 185 to an external receiving device (not shown) capable of presenting the dose size on an electronic display, e.g. a mobile phone with an application for storing and evaluating dose data.

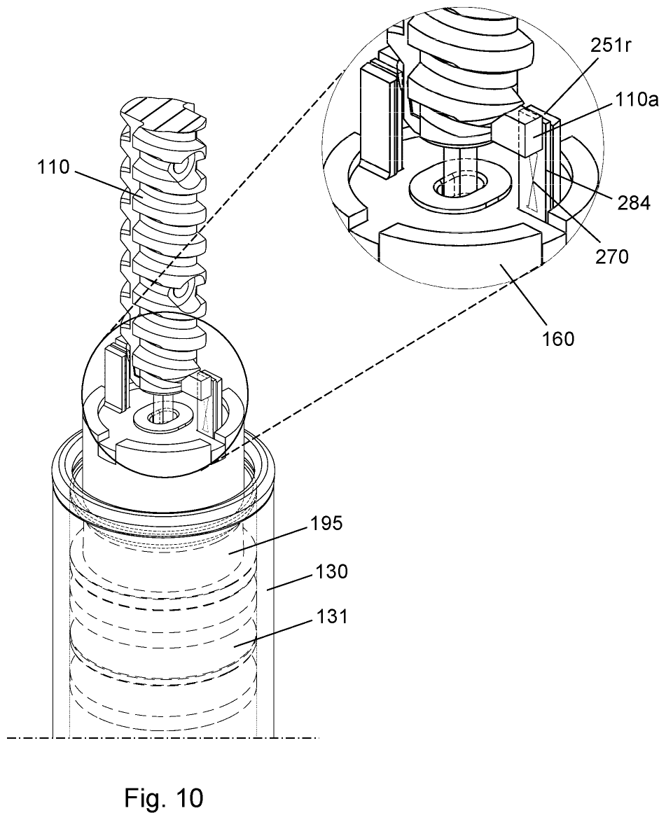

FIG. 10 illustrates a variation of the sensor module 150, where the dose estimation is based on strain gauge measurements in lieu of piezoelectric measurements. The reaction plates 151r are replaced by slightly longer, flexible reaction plates 251r to allow out-of-the-plane deformation. Correspondingly, the top sheet 181 has longer strips 284 covering respective contact surfaces of the reaction plates 251r. One, or both, of the strips 284 carries a strain gauge sensor 270 such that it is able to measure deformation of the strip 284 in question.

The radial protrusions 110a on the piston rod 110 contact the strips 284 above the strain gauge sensors 270. As the dose expelling progresses each strip 284 is in physical contact with one of the radial protrusions 110a and supported by one of the reaction plates 251r. The strain gauge sensors 270 will sense the torque variations stemming from the velocity fluctuations of the piston rod 110 as jerking bending deformations of the strips 284 and will generate a signal at each sudden deformation, indicating passage of a tooth tip by a ratchet arm (neither shown), in accordance with the above described. The generated signals are treated as previously explained.

1. A drug delivery device having a piston rod that extends along a reference axis and is forced to rotate in an expelling direction about the reference axis during a dose expelling event to cause an expelling of drug from a drug reservoir, and a resilient ratchet element is operatively coupled with the piston rod and undergoes a deflecting motion comprising elastic deformation and recovery in response to an expelling of a predetermined dose increment, which deflecting motion occasions a velocity fluctuation of the piston rod, comprising:
a rotator module operatively coupled with the piston rod and configured to rotate about the reference axis against a rotation resisting force in response to a rotation of the piston rod in the expelling direction, the rotator module comprising a sensor system for monitoring a torque transferred by the piston rod during the dose expelling event,
wherein the sensor system is configured to, from the monitored torque, identify occurrences of velocity fluctuation of the piston rod attributable to the deflecting motion of the resilient ratchet element.

2. The drug delivery device according to claim 1, further comprising a communication interface for communicating information relating to the occurrences of velocity fluctuation of the piston rod identified by the sensor system.

3. The drug delivery device according to claim 2, wherein the sensor system is further configured to estimate an expelled dose of drug from the identified occurrences of velocity fluctuation of the piston rod and the predetermined dose increment.

4. The drug delivery device according to claim 3, wherein the communication interface comprises at least one of a visual, audible and tactile structure of indicating an estimated expelled dose of drug.

5. The drug delivery device according to claim 3, wherein the communication interface comprises a transmission interface for wired or wireless transmission of an estimated expelled dose of drug to an external device.

6. The drug delivery device according to claim 1, wherein the rotator module is arranged between the piston rod and a wall portion of the drug reservoir which is displaced along the reference axis by the piston rod during the dose expelling event.

7. The drug delivery device according to claim 6, wherein the rotator module comprises a radially protruding lip structure, and the rotation resisting force is provided by a friction interface between the lip structure and an interior surface portion of the drug reservoir

8. The drug delivery device according to claim 7, wherein the rotator module comprises a first part being in rotational interlocking engagement with the piston rod and a second part including the lip structure and being rotationally coupled with the first part by a torque transmitting structure, and wherein the sensor system comprises a sensor arranged on the torque transmitting structure.

9. The drug delivery device according to claim 8, wherein the rotator module further comprises an intermediate part arranged between the first part and the second part, the intermediate part carrying the sensor system and the torque transmitting structure.

10. The drug delivery device according to claim 7, wherein the rotator module comprises a first part arranged concentrically with the piston rod and including an axially extending portion, and a second part carrying the lip structure and being rotationally locked to the first part, wherein the piston rod comprises a radial protrusion configured to apply a tangential drive force to the axially extending portion, and wherein the sensor system comprises a sensor arranged between the radial protrusion and the axially extending portion.

11. The drug delivery device according to claim 10, wherein the rotator module further comprises an intermediate part arranged substantially between the first part and the second part, the intermediate part carrying the sensor system and comprising an axially extending sensor carrier on which the sensor is arranged.

12. The drug delivery device according to claim 11, wherein the second part comprises a cup shaped wall forming a cavity, and wherein the intermediate part is arranged substantially in the cavity and the axially extending sensor carrier extends proximally therefrom.

13. The drug delivery device according to claim 8, wherein the sensor is a piezoelectric sensor.

14. A rotator module for use in a drug delivery device according to claim 1.

15. A method of detecting clicks produced by a resilient ratchet element in a drug delivery device in which a piston rod is forced to rotate about a reference axis during a dose expelling event to cause an expelling of drug and the resilient ratchet element is operatively coupled with the piston rod and undergoes a deflecting motion comprising elastic deformation and recovery in response to expelling of a predetermined dose increment, which deflecting motion occasions a velocity fluctuation of the piston rod and results in a click, the method comprising:
(i) monitoring a torque transferred by the piston rod during the dose expelling event, and
(ii) identifying, from the monitored torque, occurrences of velocity fluctuation of the piston rod attributable to the deflecting motion of the resilient ratchet element.
